(19) United States
(12) Patent Application Publication
(10) Pub. No.: US 2010/0055061 A1
(43) Pub. Date: Mar. 4, 2010

(54) COSMETIC PRODUCT WITH SMOOTHING EFFECT AND RUBBERY TEXTURE WHICH TURNS INTO POWDER WHEN APPLIED AND PROCESS FOR OBTAINING THE SAME

(75) Inventors: Antonio Mandelli, Cernusco Sul Naviglio (IT); Pietro Rando, Opera (IT)

Correspondence Address: SEED INTELLECTUAL PROPERTY LAW GROUP PLLC, 701 FIFTH AVE, SUITE 5400, SEATTLE, WA, 98104, US

(73) Assignee: INTERCOS S.P.A., Milano (IT)

(21) Appl. No.: 12/548,309

(22) Filed: Aug. 26, 2009

(30) Foreign Application Priority Data
Aug. 27, 2008 (IT) MI2008A001541

(51) Int. Cl. A61K 8/891; A61Q 1/12

(52) U.S. Cl. 424/69

(57) ABSTRACT

Described herein is a cosmetic product with smoothing effect with rubber consistency which turns into powder during application, and the process to obtain it. The cosmetic product is usable as a base for smoothing and mat effect make up.

### CROSS-REFERENCE TO RELATED APPLICATIONS

This application claims the priority benefit under 35 U.S.C. 119 of Italian Patent Application No. M12008A001541, filed Aug. 27, 2008, which is incorporated in full by reference herein.

### FIELD OF THE INVENTION

The present invention relates to a cosmetic product with smoothing effect and rubbery texture which turns into powder when applied, as well as to the process for obtaining the same. The aforesaid cosmetic product may be used as a smoothing-effect, shine-free makeup foundation.

### BACKGROUND OF THE INVENTION

A cosmetic product which uses polymethylsilsesquioxane in association with silicone oils as basic ingredient is already commercialized.

This product appears in the form of compact powder which, once applied, conceals pores, absorbs sebum in excess and reduces oiliness in excess without causing weals or leaving white residues on the skin.

Such a product has problems of stability, however, which make it unusable over time because it breaks very easily when applied.

### DETAILED DESCRIPTION OF THE INVENTION

It is the object of the present invention to provide a cosmetic product which does not have the abovementioned negative effects.

In accordance with the present invention, such an object is achieved by a cosmetic product with smoothing effect characterized in that it comprises:
from 60 to 70 % by weight of polymethylsilsesquioxane; and
from 30 to 40 % by weight of silicone oils and resins.

In particular, the polymethylsilsesquioxane used is Tospearl 3000 A PC from Momentive.

The silicone oils and resins may consist of:
from 20 to 30 % by weight of dimethicone; and
from 5 to 10 % by weight of dimethicone and dimethicone crosspolymer.

Preferably, dimethicone with low viscosity (e.g. from 10 to 350 cst) is used, such as Fluid 200/100 from Dow Corning, and dimethicone and dimethicone crosspolymer used is D.C. 9041 from the same manufacturer.

Active ingredients, solar filters, pigments, pearls and so on may be present.

The following examples are merely provided by way of non-limiting example:

### EXAMPLE 1

| Formula 1 | % by weight |
|---|---|
| Dimethicone 350 cst | 30.00 |
| Dimethicone and dimethicone crosspolymer | 5.00 |
| Polymethylsilsesquioxane | 65.00 |

### EXAMPLE 2

| Formula 2 | % by weight |
|---|---|
| Dimethicone 350 cst | 28.00 |
| Dimethicone and dimethicone crosspolymer | 7.00 |
| Polymethylsilsesquioxane | 65.00 |

### EXAMPLE 3

| Formula 3 | % by weight |
|---|---|
| Dimethicone 100 cst | 26.00 |
| Dimethicone and dimethicone crosspolymer | 9.00 |
| Polymethylsilsesquioxane | 65.00 |

### EXAMPLE 4

| Formula 4 | % by weight |
|---|---|
| Dimethicone 10 cst | 22.50 |
| Dimethicone and dimethicone crosspolymer | 10.00 |
| Polymethylsilsesquioxane | 67.00 |
| Active ingredients | 0.50 |

### EXAMPLE 5

| Formula 5 | % by weight |
|---|---|
| Dimethicone 350 cst | 30.00 |
| Dimethicone and dimethicone crosspolymer | 10.00 |
| Polymethylsilsesquioxane | 60.00 |

### EXAMPLE 6

| Formula 6 | % by weight |
|---|---|
| Dimethicone 10 cst | 20.00 |
| Dimethicone and dimethicone crosspolymer | 9.50 |
| Polymethylsilsesquioxane | 70.00 |
| Pigments and pearls | 0.50 |

The product is obtained by extruding a paste created by mixing the above-described ingredients, followed by cutting and pressing into containers.

Main features of this product are the absolute transparency thereof and the change of texture during its application, because once taken by means of a specific applicator it turns from rubbery into powder.

These features originate from the association of polymethylsilsesquioxane with silicone oils, such as dimethicone, with a viscosity in the range from 10 to 350 cst, and with silicone resins, such as for example dimethicone and dimethicone crosspolymer.

The process by which the product is obtained comprises a mixing step, an extrusion step, a cutting step and a final step of pressing into suitable containers, as described below.

The product is obtained by premixing the liquid or semi-liquid or pasty ingredients in a specific mixer equipped with planetary blades along with possible pigments and active ingredients, such as vitamins, solar filters, antioxidants.

The powder phase consisting of polymethylsilsesquioxane is then added and subjected to further mixing until an extrudable paste is formed.

The product thus obtained is in the form of a white paste which becomes transparent when it is extruded.

Once it has been cut into the desired shapes, the extruded product is put into specific containers and pressed.

What is claimed is:

1. Cosmetic product with smoothing effect characterized by comprising:
from 60 to 70% by weight of polymethylsilsesquioxane; and
from 30 to 40% by weight of silicone oils and silicone resins.

2. Cosmetic product according to claim 1, characterized in that the silicone oils and resins may consist of:
from 20 to 30% by weight of dimethicone; and
from 5 to 10% by weight of dimethicone and dimethicone crosspolymer.

3. Cosmetic product according to claim 2, characterized in that dimethicone with low viscosity is used.

4. Cosmetic product according to claims 1-3, characterized by comprising active ingredients, solar filters, pigments, pearls and so on.

5. Process for obtaining a product according to any one of claims 1-4, characterized by comprising a mixing step, an extrusion step, a cutting step and a final step of pressing into suitable containers.
